SEQUENCE LISTING (23 sequences)

SEQ ID NO: 1 - protein, 431 aa, Homo sapiens
  SIGNAL (1)...(20): Leader sequence
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTIXLPSMYNDPQFGTSCEITGFGKEXSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 2 - protein, 246 aa, Homo sapiens
IIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTIALPSMYNDPQFGTSCEITGFGKEQSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTK

SEQ ID NO: 3 - DNA, 51 bp, Artificial Sequence
  PCR primer
attaatgtcgactaaggaggtgatctaatgttaaaatttcagtgtggccaa

SEQ ID NO: 4 - DNA, 57 bp, Artificial Sequence
  PCR primer
attaataagctttcagagggccaggccattctcttccttggtgtgactcctgatcca

SEQ ID NO: 5 - DNA, 47 bp, Artificial Sequence
  PCR primer
attaattgcgcagccatcccggactatacagaccatcgccctgccct

SEQ ID NO: 6 - DNA, 50 bp, Artificial Sequence
  PCR primer
attaatgtcgactaaggaggtgatctaatgggccaaaagactctgaggcc

SEQ ID NO: 7 - DNA, 50 bp, Artificial Sequence
  PCR primer
attaatgtcgactaaggaggtgatctaatgaagactctgaggccccgctt

SEQ ID NO: 8 - DNA, 50 bp, Artificial Sequence
  PCR primer
attaatgtcgactaaggaggtgatctaatgattattgggggagaattcac

SEQ ID NO: 9 - DNA, 56 bp, Artificial Sequence
  PCR primer
attaatgtcgactaaggaggtgatctaatgattgggggagaattcaccaccatcga

SEQ ID NO: 10 - DNA, 39 bp, Artificial Sequence
  PCR primer
attaataagctttcactcttccttggtgtgactcctgat

SEQ ID NO: 11 - DNA, 39 bp, Artificial Sequence
  PCR primer
attaataagctttcattccttggtgtgactcctgatcca

SEQ ID NO: 12 - DNA, 40 bp, Artificial Sequence
  PCR primer
attaataagctttcacttggtgtgactcctgatccagggt

SEQ ID NO: 13 - DNA, 46 bp, Artificial Sequence
  PCR primer
attaatcagctgctccggatagagatagtcggtagactgctctttt

SEQ ID NO: 14 - DNA, 28 bp, Artificial Sequence
  PCR primer
attaatcagctgaaaatgactgttgtga

SEQ ID NO: 15 - DNA, 51 bp, Artificial Sequence; identical to SEQ ID NO: 3
  PCR primer

SEQ ID NO: 16 - DNA, 37 bp, Artificial Sequence
  PCR primer
attaatgctagcctcgagccaccatgagagccctgct

SEQ ID NO: 17 - DNA, 42 bp, Artificial Sequence
  PCR primer
attaatgctagcctcgagtcacttgttgtgactgcggatcca

SEQ ID NO: 18 - DNA, 44 bp, Artificial Sequence
  PCR primer
ggtggtgaattctcccccaataatgcctttggagtcgctcacga

SEQ ID NO: 19 - DNA, 111 bp, Artificial Sequence
  PCR primer
atgttctctccaattttgtccttggaaattattttagctttggctactttgcaatctgtcttcgctcagccagttatctgcactaccgttggttccgctgccgagggatcc

SEQ ID NO: 20 - DNA, 22 bp, Artificial Sequence
  PCR primer
gaaacttccaaaagtcgccata

SEQ ID NO: 21 - DNA, 92 bp, Artificial Sequence
  PCR primer
attaatgaattcctcgagcggtccgggatccctcggcagcggaaccaacggtagtgcagataactggctgagcgaagacagattgcaaagta

SEQ ID NO: 22 - DNA, 46 bp, Artificial Sequence
  PCR primer
attaatggatccttggacaagaggattattgggggagaattcacca

SEQ ID NO: 23 - DNA, 47 bp, Artificial Sequence
  PCR primer
attaatctcgagcggtccgtcacttggtgtgactgcgaatccagggt

(19) United States
(12) Patent Application Publication
(10) Pub. No.: US 2002/0106775 A1
(43) Pub. Date: Aug. 8, 2002

(54) HIGHLY CRYSTALLINE UROKINASE

(76) Inventors: JIEYI WANG, GURNEE, IL (US); VICKI L. NIENABER, GURNEE, IL (US); JACK HENKIN, HIGHLAND PARK, IL (US); RICHARD A. SMITH, LAKE BLUFF, IL (US); KARL A. WALTER, LAKE BLUFF, IL (US); JEAN M. SEVERIN, WADSWORTH, IL (US); ROHINTON EDALJI, WADSWORTH, IL (US); ROBERT W. JOHNSON JR., GURNEE, IL (US); THOMAS F. HOLZMAN, LIBERTYVILLE, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 09/264,468

(22) Filed: Mar. 5, 1999

(51) Int. Cl. C12P 21/06

(52) U.S. Cl. 435/215; 435/183; 435/320.1; 435/462; 530/350; 536/23.2

(57) ABSTRACT

The present disclosure describes a biologically active modified urokinase and high resolution crystalline forms of modified urokinase. Polynucleotides which encode modified urokinase and methods for making modified urokinase are also disclosed.

This application claims priority to U.S. application Ser. No. 09/036,361 filed Mar. 6, 1998.

### TECHNICAL FIELD

The present invention relates to polypeptides, crystalline forms of those polypeptides and polynucleotides encoding the polypeptides. More specifically, the invention relates to a modified urokinase capable of forming high resolution crystals, as well as polynucleotides which encode modified urokinase and methods for producing modified urokinase.

### BACKGROUND OF THE INVENTION

Urinary plasminogen activator (uPA, also known as urokinase or UK) is a highly specific serine protease which converts plasminogen to plasmin by catalyzing the cleavage of a single peptide bond (L. Summaria et al., J. Biol. Chem., 242(19): 4279-4283 [1967]). UPA is secreted by cells as 411-amino acid single chain zymogen termed pro-urokinase (pro-UK) or pro-uPA. Activation of pro-uPA requires enzymatic cleavage at the Lys¹⁵⁸-Ile¹⁵⁹ bond. The active (i.e. cleaved) protein contains an N-terminal “A-chain” (amino acid residues 1-158 of SEQ ID NO: 1) and C-terminal “B-chain” (amino acid residues 159-411) which are joined via a disulfide bond at Cys residues 148 and 279 (W. A. Guenzler et al., Hoppe-Seyler's Z. Physiol. Chem. Bd. 363, S133-141 [1982]). The uPA A-chain comprises a triple disulfide region of about 40 amino acid residues called the “growth factor domain” and a larger triple disulfide kringle. B-chain comprises the serine protease domain having the catalytic triad (i.e. His²⁰⁴, Ser³⁵⁶, and Asp³⁵⁰) typical of serine proteases. UPA also possesses a glycosylation site at amino acid residue 302.

UPA is responsible for plasminogen activation on cell surfaces and is unique in having its own high affinity receptor, uPAR, which greatly enhances its action on plasminogen absorbed to cells. The uPAR also focalizes to cell-cell junctions and to the leading edges of invading cells. Thus, uPA is positioned spatially and metabolically to play a pivotal role in the directed cascade of protease activity needed for cancer invasion and metastasis, and angiogenesis. Elevated uPA and/or uPAR is strongly associated with malignant tissue, and with poor clinical prognosis in cancer. There is substantial evidence from tumor cell invasion and animal metastasis studies to suggest that blocking uPA will slow the growth and metastasis of tumors and their elicitation of the blood supply. Thus, inhibitors which interact with the ligand binding domain (LBD) at the urokinase protein active site and block introduction of the natural substrate to the LBD could be useful therapeutically in the treatment of these conditions.

It is well established that single crystal X-ray diffraction allows experimental determination of protein structures at the atomic level and integration of these protein structures into the drug discovery process. A three dimensional structure of a protein permits identification of the LBD at a protein active site. Additionally, identification of a ligand's relation to binding clefts and/or functionality at the LBD may be elucidated by co-crystallizing the ligand with the protein and used to evaluate the potential effectiveness of the ligand, in this case a drug candidate, as an enzyme inhibitor, agonist, or antagonist. Co-crystal structures indicate which sites of the drug candidate should or should not be derivatized as well as the nature and size of functional groups most likely to result in increased potency, i.e., better binding at the LBD.

The best operating mode of structure-directed drug discovery requires a high-quality protein crystal which has an accessible, empty binding site and which reproducibly diffracts to high resolution (<2.0 Å). As is well known in the art, an empty binding site permits introduction of the ligand of interest into the LBD while the protein is crystalline, and high resolution diffraction permits accurate identification of ligand interaction with the LBD.

A low molecular weight urokinase-type plasminogen activator-inhibitor complex is known in the art (Spraggon et al., Structure 3: 681-691 [1995]). The data obtained, however, were of low resolution (3.1 Å), and the crystal contained irreversibly-bound inhibitor at the LBD. Attempts to incorporate other inhibitors with the LBD using co-crystallizing methodology have provided only low-quality crystals.

Thus there is a need for high-quality urokinase crystals from which ligand-binding data can be gathered.

### BRIEF DESCRIPTION OF THE FIGURES

FIG. 1 shows the amino acid sequence (SEQ ID NO: 1) of human urinary-type plasminogen (uPA) with the modification that the amino acid residues at positions 279 and 302 are indicated by Xaa. In native uPA, Xaa at amino acid position 279 is Cys and at amino acid position 302 is Asn. (In SEQ ID NO: 1, residues −1 to −20 represent the native leader sequence of human uPA).

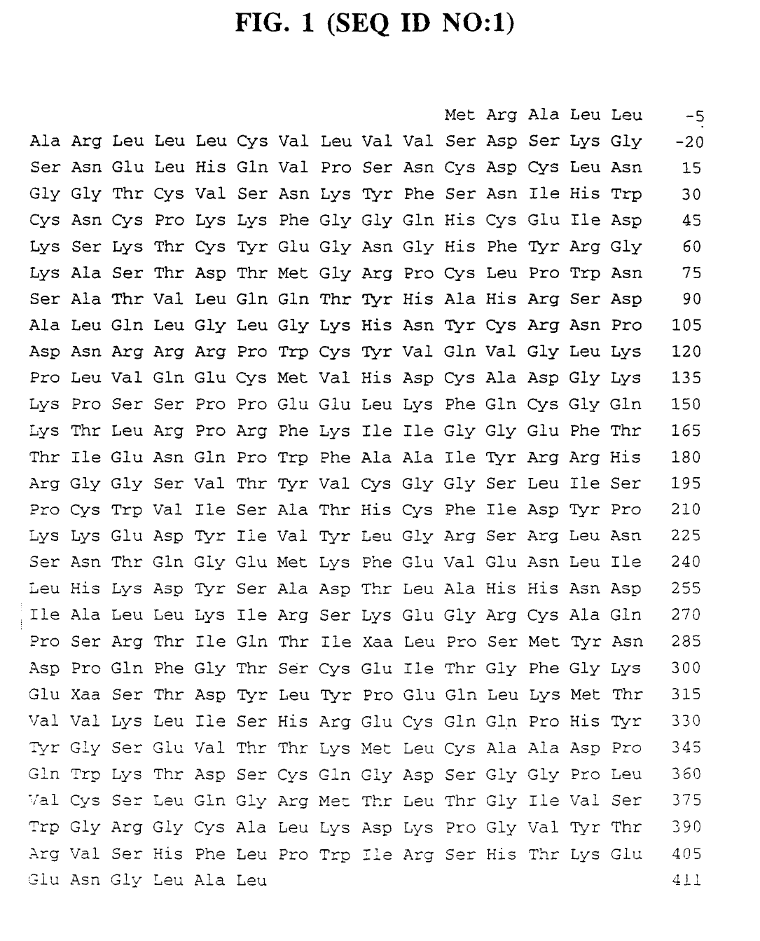

FIG. 2 shows the amino acid sequence (SEQ ID NO: 2) of a preferred polypeptide.

### SUMMARY OF THE INVENTION

The present invention provides a polynucleotide(s) which encodes a biologically active modified urinary-type plasminogen activator (mod-uPA) having at least 70% identity to an amino acid sequence selected from the group consisting of (a) amino acid position 159 to amino acid position 404 of SEQ ID NO: 1; (b) amino acid position 159 to amino acid position 405 of SEQ ID NO: 1; (c) amino acid position 159 to amino acid position 406 of SEQ ID NO: 1; (d) amino acid position 159 to amino acid position 407 of SEQ ID NO: 1; (e) amino acid position 159 to amino acid position 408 of SEQ ID NO: 1; (f) amino acid position 159 to amino acid position 409 of SEQ ID NO: 1; (g) amino acid position 159 to amino acid position 410 of SEQ ID NO: 1; and (h) amino acid position 159 to amino acid position 411 of SEQ ID NO: 1; wherein in (a)-(h) above, the amino acid residues designated as Xaa at position 279 (Xaa²⁷⁹) and position 302 (Xaa³⁰²) can be any amino acid. In a preferred embodiment, the Xaa residue at position 279 is Ala. In another preferred embodiment, the Xaa residue at position 302 is Gln. In an even more preferred embodiment, the Xaa residues at positions 279 and 302 are Ala and Gln, respectively.

In another embodiment, the invention provides a recombinant vector comprising a polynucleotide as described above. In a preferred embodiment, the vector comprises one of the above-described polynucleotide having Ala at Xaa residue 279 and Gln at Xaa residue 302. The invention further provides host cells comprising the recombinant vectors.

In yet another embodiment, the invention provides a biologically active non-glycosylated modified urinary-type plasminogen activator (mod-uPA) having at least 70% identity to an amino acid sequence selected from the group consisting of (a) amino acid position 159 to amino acid position 404 of SEQ ID NO: 1; (b) amino acid position 159 to amino acid position 405 of SEQ ID NO: 1; (c) amino acid position 159 to amino acid position 406 of SEQ ID NO: 1; (d) amino acid position 159 to amino acid position 407 of SEQ ID NO: 1; (e) amino acid position 159 to amino acid position 408 of SEQ ID NO: 1; (f) amino acid position 159 to amino acid position 409 of SEQ ID NO: 1; (g) amino acid position 159 to amino acid position 410 of SEQ ID NO: 1; and (h) amino acid position 159 to amino acid position 411 of SEQ ID NO: 1; with the proviso that when said mod-uPA is glycosylated, residue 279 is any amino acid residue other than Cys and when said mod-uPA is non-glycosylated, residue 279 is any amino acid. A preferred mod-uPA is one in which the Xaa residue at position 279 is Ala. A more preferred mod-uPA is one in which the Xaa residue at position 302 is Gln. In an even more preferred embodiment, the Xaa residues at positions 279 and 302 are Ala and Gln, respectively. In another embodiment, the invention provides a crystalline form of mod-uPA wherein the primary structure of said mod-uPA has the structure of a polypeptide described above. The primary structure of the crystalline form also has the preferred embodiments described above.

The invention further provides a method for making mod-uPA comprising the steps of: (a) culturing the host cell of the invention under conditions that allow the production of the mod-uPA polypeptide; and (b) recovering the mod-uPA polypeptide.

### DETAILED DESCRIPTION OF THE INVENTION

The practice of the present invention will employ, unless otherwise indicated, conventional techniques of molecular biology, microbiology and recombinant DNA technology, which are within the skill of the ordinary artisan. Such techniques are explained fully in the literature. See, e.g. Sambrook, Fritsch & Maniatis, Molecular Cloning: A Laboratory Manual, Second Edition (1989); DNA Cloning, Vols, I and II (D. N. Glover ed. 1985); the series, Methods in Enzymology (S. Colowick and N. Kaplan eds., Academic Press, Inc.); Scopes, Protein Purification: Principles and Practice (2nd ed., Springer-Verlag); and PCR: A practical Approach (McPherson et al. eds (1991) IRL Press).

All patents, patent applications and publications cited herein, whether supra or infra, are hereby incorporated by reference in their entirety.

As used in this specification and the appended claims, the singular forms “a”, “an”, and “the” include plural references unless the content clearly dictates otherwise.

I. Definitions

In describing the present invention, the following terms will be employed and are intended to be defined as indicated below:

The term “polynucleotide” as used herein refers to a polymeric form of nucleotides of any length, either ribonucleotides or deoxyribonucleotides. The term refers only to the primary structure of the molecule. Thus, the term includes double- and single-stranded DNA, as well as double- and single-stranded RNA. It also includes modifications, such as by methylation and/or by capping, and unmodified forms of the polynucleotide.

“Polypeptide” and “protein” are used interchangeably herein and indicate a molecular chain of amino acids linked through peptide bonds. The terms do not refer to a specific length of the product. Thus, peptides and oligopeptides are included within the definition of polypeptide. This term is also intended to refer to post-translational modifications of the polypeptide, for example, glycosylations, acetylations, phosphorylations and the like. In addition, protein fragments, analogs, muteins, fusion proteins and the like are included within the meaning of polypeptide. Polypeptides and proteins of the invention may be made by any means known to those of ordinary skill in the art (i.e. they may be isolated or made by recombinant, synthetic or semi-synthetic techniques).

As used herein, the term “analogue” refers to a polypeptide which demonstrates like biological activity to disclosed mod-uPA polypeptides provided herein. It is well known in the art that modifications and changes can be made without substantially altering the biological function of a polypeptide. In making such changes, substitutions of like amino acid residues can be made on the basis of relative similarity of side-chain substituents, for example, their size, charge, hydrophobicity, hydrophilicity and the like. Alterations of the type described may be made to enhance the polypeptide's potency or stability to enzymatic breakdown or pharmacokinetics. Thus, sequences deemed as within the scope of the invention, include those analogous sequences characterized by a change in amino acid residue sequence or type wherein the change does not alter the fundamental nature and biological activity of the aforementioned.

In general, “similarity” means the exact amino acid to amino acid comparison of two or more polypeptides at the appropriate place, where amino acids are identical or possess similar chemical and/or physical properties such as charge or hydrophobicity. “Percent similarity” can be determined between the compared polypeptide sequences using techniques well known in the art. In general, “identity” refers to an exact nucleotide to nucleotide or amino acid to amino acid correspondence of two polynucleotides or polypeptide sequences, respectively. Two or more polynucleotide sequences can be compared by determining their “percent identity.” Two amino acid sequences likewise can be compared by determining their “percent identity.”

The techniques for determining nucleic acid and amino acid sequence identity as well as amino acid sequence similarity are well known in the art. For example, one method for determining nucleic acid and amino acid sequence identity includes determining the nucleotide sequence of the mRNA for that gene (usually via a cDNA intermediate) and determining the amino acid sequence encoded therein, and comparing this to a second amino acid sequence. The programs available in the Wisconsin Sequence Analysis Package (available from Genetics Computer Group, Madison, Wis.), for example, the GAP program (with default or other parameters), are capable of calculating both the identity between two polynucleotides and the identity and similarity between two polypeptide sequences, respectively. Other programs for calculating identity or similarity between sequences are also known in the art.

The term “degenerate variant” or “structurally conserved mutation” refers to a polynucleotide containing changes in the nucleic acid sequence thereof, such as insertions, deletions or substitutions, that encodes a polypeptide having the same amino acid sequence as the polypeptide encoded by the polynucleotide from which the degenerate variant is derived.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, immaterial of the method by which the DNA is introduced into the cell or the subsequent disposition of the cell. These terms include the progeny of the original cell which has been transfected. As used herein “replicon” means any genetic element, such as a plasmid, a chromosome or a virus, that behaves as an autonomous unit of polynucleotide replication within a cell.

A “vector” is a replicon in which another polynucleotide segment is attached, such as to bring about the replication and/or expression of the attached segment. The term includes expression vectors, cloning vectors and the like.

The term “control sequence” refers to polynucleotide sequence which effects the expression of coding sequences to which it is ligated. The nature of such control sequences differs depending upon the host organism. In prokaryotes, such control sequences generally include a promoter, a ribosomal binding site and a terminator; in eukaryotes, such control sequences generally include a promoter, terminator and, in some instances, enhancers. The term “control sequence” thus is intended to include at a minimum all components whose presence is necessary for expression, and also may include additional components whose presence is advantageous, for example, leader sequences.

A “coding sequence” is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Mutants or analogs may be prepared by the deletion of a portion of the coding sequence, by insertion of a sequence, and/or by substitution of one or more nucleotides within the sequence. Techniques for modifying nucleotide sequences, such as site-directed mutagenesis, are well known to those skilled in the art. See, e.g., Sambrook, et al., supra; DNA Cloning, Vols, I and II, supra; Nucleic Acid Hybridization, supra.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences. The coding sequence may be operably linked to control sequences that direct the transcription of the polynucleotide whereby said polynucleotide is expressed in a host cell.

The term “open reading frame” or “ORF” refers to a region of a polynucleotide sequence which encodes a polypeptide; this region may represent a portion of a coding sequence or a total coding sequence.

The term “transformation” refers to the insertion of an exogenous polynucleotide into a host cell, irrespective of the method used for the insertion, or the molecular form of the polynucleotide that is inserted. For example, injection, direct uptake, transduction, and f-mating are included. Furthermore, the insertion of a polynucleotide per se and the insertion of a plasmid or vector comprising the exogenous polynucleotide are included. The exogenous polynucleotide may be directly transcribed and translated by the cell, maintained as a non-integrated vector, for example, a plasmid, or alternatively, may be integrated into the host genome.

The term “isolated” as used herein means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence complementary to a target nucleotide sequence and used to hybridize to the target nucleotide sequence and serve as an initiation point for nucleotide polymerization catalyzed by either DNA polymerase, RNA polymerase or reverse transcriptase.

A “recombinant polypeptide” as used herein means at least a polypeptide which by virtue of its origin or manipulation is not associated with all or a portion of the polypeptide with which it is associated in nature and/or is linked to a polypeptide other than that to which it is linked in nature. A recombinant or derived polypeptide is not necessarily translated from a designated nucleic acid sequence. It also may be generated in any manner, including chemical synthesis or expression of a recombinant expression system.

The term “synthetic peptide” as used herein means a polymeric form of amino acids of any length, which may be chemically synthesized by methods well-known to an ordinarily skill practitioner. These synthetic peptides are useful in various applications.

“Purified polynucleotide” refers to a polynucleotide of interest or fragment thereof which is essentially free, i.e., contains less than about 50%, preferably less than about 70%, and more preferably, less than about 90% of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density. Thus, “purified polypeptide” means a polypeptide of interest or fragment thereof which is essentially free, that is, contains less than about 50%, preferably less than about 70%, and more preferably, less than about 90% of cellular components with which the polypeptide of interest is naturally associated. Methods for purifying are known in the art.

“Purified product” refers to a preparation of the product which has been isolated from the cellular constituents with which the product is normally associated.

II. Reagents

a. Polypeptides: The present invention provides a modified urokinase polypeptide (hereinafter termed “mod-uPA”) comprising an amino acid sequence selected from the group consisting of

(a) amino acid position 159 to about amino acid position 404 of SEQ ID NO: 1;

(b) amino acid position 159 to amino acid position 405 of SEQ ID NO: 1;

(c) amino acid position 159 to amino acid position 406 of SEQ ID NO: 1;

(d) amino acid position 159 to amino acid position 407 of SEQ ID NO: 1;

(e) amino acid position 159 to amino acid position 408 of SEQ ID NO: 1;

(f) from amino acid position 159 to amino acid position 409 of SEQ ID NO: 1;

(g) amino acid position 159 to amino acid position 410 of SEQ ID NO: 1;

(h) amino acid position 159 to amino acid position 411 of SEQ ID NO: 1;

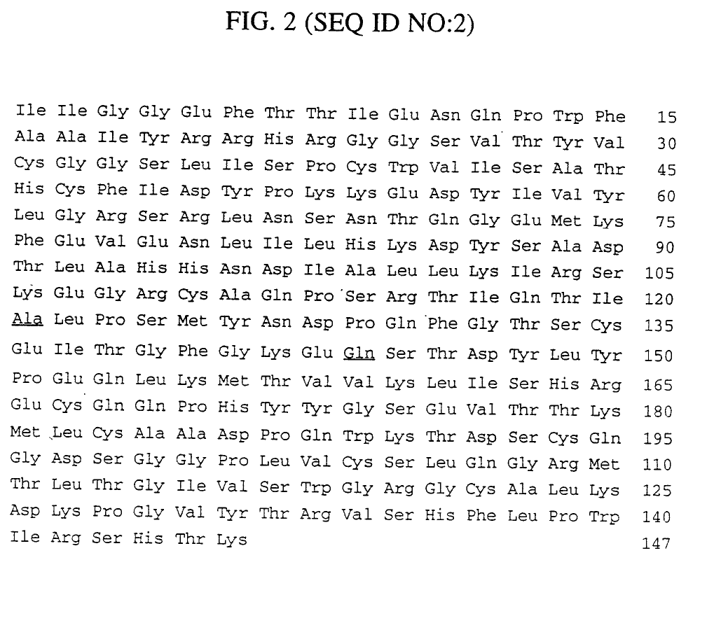

with the proviso that when said mod-uPA is glycosylated, residue 279 (Xaa²⁷⁹) is any amino acid residue other than Cys and when said mod-uPA is non-glycosylated, residue 279 is any amino acid and wherein the polypeptide has like biological activity, (e.g. catalytic and/or immunological activity) to human urokinase. In a preferred embodiment shown in FIG. 2 (SEQ ID NO: 2), Xaa²⁷⁹ is Ala and Xaa³⁰² is Gln. Polypeptides of the invention also include analogs and mutated or variant proteins of SEQ ID NO: 1 that retain such activity. Generally, a polypeptide analog of mod-uPA will have at least about 60% identity, preferably about 70% identity, more preferably about 75-85% identity, even more preferably about 90% identity and most preferably about 95% or more identity to (a)-(h) above. Thus, included within the scope of the invention are polypeptides in which one or more of the amino acid residues is substituted with a conserved or non-conserved amino acid residue (preferably a conserved amino acid residue) and such substituted amino acid residue may or may not be one encoded by the genetic code. Since it is known in the art that residues His²⁰⁴, Asp²⁵⁵, Asp³⁵⁰, and Ser³⁵⁶ (as well as all other cysteine residues in the B chain with the exception of Cys²⁷⁹) are necessary to preserve biological activity, one of ordinary skill in the art can readily ascertain the various residues which can be altered without affecting the activity of the resulting mod-uPA.

A “conservative change” is one typically in the range of about 1 to 5 amino acids, wherein the substituted amino acid has similar structural or chemical properties, e.g., replacement of leucine with isoleucine or threonine with serine. In contrast, a nonconservative change is one in which the substituted amino acid differs structurally or chemically from the original residue, e.g. replacement of a glycine with a tryptophan. Similar minor variations may also include amino acid deletions or insertions or both. Guidance in determining which and how many amino acid residues may be substituted, inserted or deleted without changing biological or immunological activity may be found using computer programs well known in the art, for example, DNASTAR software (DNASTAR Inc., Madison, Wis.).

The invention further provides for any of the aforementioned polypeptides in which one or more of the amino acid residues includes a substituent group; or is fused with another compound, such as a compound to increase the half-life of the polypeptide (for example, polyethylene glycol); or it may be one in which the additional amino acids are fused to the polypeptide, such as a leader or secretory sequence or a sequence which is employed for purification of the polypeptide or a proprotein sequence. Furthermore, a polypeptide of the invention may or may not be glycosylated.

Polypeptides of the invention may be made by any means known to those of ordinary skill in the art such as by isolation or by recombinant, synthetic or semi-synthetic techniques. Furthermore, as will be apparent to those of ordinary skill in the art, the type of residue selected for Xaa positions 279 and 302 as well as the manner of making the polypeptide will depend upon whether the polypeptide is to be glycosylated or not. For example, when a non-glycosylated, recombinantly made polypeptide of the invention is desired, the user may select any amino acid for Xaa²⁷⁹ and Xaa³⁰². Furthermore, in this case, the user must select a recombinant host (such as a procaryotic host) which does not glycosylate proteins. In contrast, when a user desires a polypeptide of the invention to be glycosylated, then the amino acid residue at Xaa²⁷⁹ must be one other than Cys. In this situation, one desiring to produce the protein by recombinant techniques (i.e. via a recombinant polynucleotide construct) will know to express that construct in a host cell which glycosylates proteins (for example, a eucaryotic cell such as Pichia) and not in a procaryotic cell, such as E. coli, which will not glycosylate the protein. Furthermore, when a recombinantly generated polypeptide is to be made from native human uPA, and is intended to contain Cys²⁷⁹, the polynucleotide construct which encodes the human uPA must be modified so as to prevent the formation of a disulfide bond between Cys¹⁴⁸ and Cys²⁷⁹. To achieve this result, one must prepare a construct that is modified at the Cys 148 residue (Cys¹⁴⁸) of SEQ ID NO: 1. In addition, such a construct must be expressed in a host cell that does not glycosylate the protein. As will also be apparent to those of ordinary skill in the art, one desiring to make a protein of the invention in this manner, must cleave the A chain from the B chain either in vitro or in vivo.

Conversely, when the recombinantly generated polypeptide is to be generated from native uPA and is intended to have a non-Cys residue at position 279 of SEQ ID NO: 1, one must generate a polynucleotide construct that is modified at the Cys²⁷⁹ position but may leave the Cys¹⁴⁸ position unaffected. Methods for generating this and other mutations are considered within the skill limit of the routine practitioner as well as all other techniques for producing the polypeptides as described hereinabove.

The present invention also provides high resolution crystalline forms of the polypeptides described herein. Methods of making crystalline forms of polypeptides of the invention are well known (see for example, U.S. Pat. No. 4,886,646, issued December 12) and are considered as within the skill level of the routine practitioner. Thus, using the polypeptides, polynucleotides and methodologies described herein, a sufficient amount of a recombinant polypeptide of the present invention may be made available to generate high resolution crystals to perform analytical studies such as X-ray crystallography.

b. Polynuceleotides: The present invention also provides reagents such as polynucleotides which encode the biologically active mod-uPA polypeptides described above. A polynucleotide of the invention may be in the form of mRNA or DNA. DNAs in the form of cDNA, genomic DNA, and synthetic DNA are within the scope of the present invention. The DNA may be double-stranded or single-stranded, and if single stranded may be the coding (sense) strand or non-coding (anti-sense) strand. The coding sequence which encodes the polypeptide may be identical to the coding sequence provided herein or may be a different coding sequence which coding sequence, as a result of the redundancy or degeneracy of the genetic code, encodes the same polypeptide as the DNA provided herein. A preferred polynucleotide is SEQ ID NO: 2 (shown in FIG. 2). The sequences disclosed herein represent unique polynucleotides which can be used for making and purifying mod-uPA.

A polynucleotide of the invention may include only the coding sequence for the polypeptide, or the coding sequence for the polypeptide and additional coding sequence such as a leader or secretory sequence or a proprotein sequence, or the coding sequence for the polypeptide (and optionally additional coding sequence) and non-coding sequence, such as a non-coding sequence 5′ and/or 3′ of the coding sequence for the polypeptide.

In addition, the invention includes variant polynucleotides containing modifications such as polynucleotide deletions, substitutions or additions; and any polypeptide modification resulting from the variant polynucleotide sequence. A polynucleotide of the present invention also may have a coding sequence which is a naturally occurring allelic variant of the coding sequence provided herein.

In addition, the coding sequence for the polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and will have the leader sequence cleaved by the host cell to form the polypeptide. Thus, the polynucleotide of the present invention may encode for a protein, or for a protein having a presequence (leader sequence).

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein. See, for example, I. Wilson, et al., Cell 37:767 (1984). A variety of expression vectors are commercial available for this purpose and are intended as within the scope of the invention.

It is contemplated that polynucleotides will be considered to hybridize to the sequences provided herein if there is at least 50%, and preferably at least 70% identity between the polynucleotide and the sequence.

III. Recombinant Technology

The present invention provides host cells and expression vectors comprising polynucleotides of the present invention and recombinant methods for the production of polypeptides they encode. Such methods comprise culturing the host cells under conditions suitable for the expression of the mod-uPA polynucleotide and recovering a mod-uPA polypeptide from the cell culture.

The polynucleotide(s) of the present invention may be employed for producing a polypeptide(s) by recombinant techniques. Thus, the polynucleotide sequence may be included in any one of a variety of expression vehicles, in particular vectors or plasmids for expressing a polypeptide. Such vectors include chromosomal, nonchromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus, and pseudorabies. In a preferred aspect of this embodiment, the vector further comprises regulatory sequences, including, for example, a promoter, operably linked to the sequence.

The appropriate DNA sequence may be inserted into the vector by a variety of procedures. In general, the DNA sequence is inserted into appropriate restriction endonuclease sites by procedures known in the art. Such procedures and others are deemed to be within the scope of those skilled in the art Large numbers of suitable vectors and promoters are known to those of skill in the art and are commercially available. The following vectors are provided by way of example. Bacterial: pSPORT1 (Life Technologies, Gaithersburg, Md.), pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia). Also, appropriate cloning and expression vectors for use with prokaryotic and eukaryotic hosts are described by Sambrook et al., supra.

The expression vector(s) containing the appropriate DNA sequence as hereinabove described, may be employed to transform an appropriate host to permit the host to express the protein. Host cells are genetically engineered (transduced or transformed or transfected) with the vectors of this invention which may be a cloning vector or an expression vector. For example, introduction of such constructs into a host cell can be effected by calcium phosphate transfection, DEAE-Dextran mediated transfection, or electroporation (L. Davis et al., “Basic Methods in Molecular Biology”, 2nd edition, Appleton and Lang, Paramount Publishing, East Norwalk, Conn. (1994)). The engineered host cells can be cultured in conventional nutrient media modified as appropriate for activating promoters and selecting transformants. The culture conditions, such as temperature, pH and the like, are those previously used with the host cell selected for expression, and will be apparent to the ordinarily skilled artisan. The selection of an appropriate host is deemed to be within the scope of those skilled in the art from the teachings provided herein.

In a further embodiment, the present invention provides host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Representative examples of appropriate hosts include bacterial cells, such as E. coli, Salmonella typhimurium; Streptomyces sp.; yeast cells such as Pichia sp.; insect cells such as Drosophila and Sf9; animal cells such as CHO, COS or Bowes melanoma; plant cells, etc.

The constructs in host cells can be used in a conventional manner to produce the gene product encoded by the recombinant sequence. Proteins can be expressed in mammalian cells, yeast, bacteria, or other cells under the control of appropriate promoters. Cell-free translation systems can also be employed to produce such proteins using RNAs derived from the DNA constructs of the present invention. Alternatively, the polypeptides of the invention can be synthetically produced by conventional peptide synthesizers.

Following transformation of a suitable host strain and growth of the host strain to an appropriate cell density, the selected promoter is derepressed by appropriate means (e.g., temperature shift or chemical induction), and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well-known to the ordinary artisan.

Mod-uPA polypeptide is recovered and purified from recombinant cell cultures by known methods including ammonium sulfate or ethanol precipitation, acid extraction, anion or cation exchange chromatography, phosphocellulose chromatography, hydrophobic interaction chromatography, hydroxyapatite chromatography or lectin chromatography. It is preferred to have low concentrations (approximately 0.1-5 mM) of calcium ion present during purification (Price, et al., J. Biol. Chem. 244:917 [1969]). Protein refolding steps can be used, as necessary, in completing configuration of the protein. Finally, high performance liquid chromatography (HPLC) can be employed for final purification steps.

III. Drug Design

The goal of rational drug design is to produce structural analogs of biologically active polypeptides of interest or of the small molecules including agonists, antagonists, or inhibitors with which they interact. Such structural analogs can be used to fashion drugs which are more active or stable forms of the polypeptide or which enhance or interfere with the function of a polypeptide in vivo. (see J. Hodgson, Bio/Technology 9:19-21 (1991)).

For example, in one approach, the three-dimensional structure of a crystalline polypeptide, or of a polypeptide-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide may be gained by modeling based on the structure of homologous proteins. In both cases, relevant structural information is used to design analogous polypeptide-like molecules or to identify efficient inhibitors.

Useful examples of rational drug design may include molecules which have improved activity or stability as shown by S. Braxton et al., Biochemistry 31:7796-7801 (1992), or which act as inhibitors, agonists, or antagonists of native peptides as shown by S. B. P. Athauda et al., J. Biochem. (Tokyo) 113 (6):742-746 (1993).

Having now generally described the invention, a complete understanding can be obtained by reference to the following specific examples. The following examples are given for the purpose of illustrating various embodiments of the invention and are not intended to limit the present invention in any fashion.

### EXAMPLE 1

### Mutagenesis Analysis of uPA

Mutants of human uPA were cloned into a dicistronic bacterial expression vector pCFK12 (Pilot-Matias, T. J. et al., Gene 128: 219-225 [1993]). The following oligo nucleotides were used to generate various uPA mutants by PCR:

| | |
|---|---|
| SEQ ID NO: | SEQUENCE OF PCR PRIMER |

| | |
|---|---|
| 3 | 5′-ATTAATGTCGACTAAGGAGGTGATCTAATGTTAATTTCAGTGTGGCCAA-3′ |
| 4 | 5′-ATTAATAAGCTTTCAGAGGGCCAGGCCATTCTCTTCCTTGGTGTGACTCCTGATCCA-3′ |
| 5 | 5′-ATTAATTGCGCAGCCATCCCGGACTATACAGACCATCGCCCTGCCCT-3′ |
| 6 | 5′-ATTAATGTCGACTAAGGAGGTGATCTAATGGGCCAAAAGACTCTGAGGCC-3′ |
| 7 | 5′-ATTAATGTCGACTAAGGAGGTGATCTAATGAAGACTCTGAGGCCCCGCTT-3′ |
| 8 | 5′-ATTAATGTCGACTAAGGAGGTGATCTAATGATTATTGGGGGAGAATTCAC-3′ |
| 9 | 5′-ATTAATGTCGACTAAGGAGGTGATCTAATGATTGGGGGAGAATTCACCACCATCGA-3′ |
| 10 | 5′-ATTAATAAGCTTTCACTCTTCCTTGGTGTGACTCCTGAT-3′ |
| 11 | 5′-ATTAATAAGCTTTCATTCCTTGGTGTGACTCCTGATCCA-3′ |
| 12 | 5′-ATTAATAAGCTTTCACTTGGTGTGACTCCTGATCCAGGGT-3′ |

The initial cloning of a low molecular weight uPA, hereinafter designated LMW-uPA (L144-L411) was performed using human uPA cDNA as template and SEQ ID NOs: 3 and 4 as primers in a standard PCR reaction. (The nucleic acid and protein sequence of human uPA can be found in U.S. Pat. No. 5,112,755, issued May 12, 1992). The PCR amplified DNA was gel purified and digested with restriction enzymes SalI and HindIII. The digested product then was ligated into a pBCFK12 vector previously cut with the same two enzymes to generate expression vector pBC-LMW-uPA. The vector was transformed in DH5α cells (Life Technologies, Gaithersburg, Md.), isolated and the sequence confirmed by DNA sequencing. The production of LMW-UPA in bacteria was analyzed by SDS-PAGE and zymography (Granelli-Pipemo, A. and Reich.E., J. Exp. Med., 148: 223-234, (1978)), which measures plasminogen activation by uPA.. LMW-UPA(L144-L411) was expressed in E. coli as shown on a commassie blue stained gel, and was active in the zymographic assay.

The success of the quick expression and detection of LMW-uPA in E. coli made it possible to perform mutagenesis analysis of uPA in order to determine its minimum functional structure. One mutant having a Cys²⁷⁹ to Ala²⁷⁹ replacement was made with SEQ ID Nos: 4 and 5 by PCR. The PCR product was cut with AviII and Hind III, and used to replace a AviII and HindIII fragment in the pBC-LMW-uPA construct. The resulting LMW-uPA-A²⁷⁹ construct was expressed in E. coli and the product shown to be active in zymography (data not shown). Using the oligonucleotides designated below further mutants with N- or C-terminal truncations were generated by PCR:

| | Characteristics of Mutants Relative to | SEQ ID |
|---|---|---|
| Mutant | LMW-uPA | NOs: |
| LMW-uPA-N 5 | 5 amino acid deletion from the | 6 and 2 |
| | N-terminus | |
| LMW-uPA-N 7 | 7 amino acid deletion from the | 7 and 2 |
| | N-terminus | |
| LMW-uPA-N 15 | 15 amino acid deletion from the | 8 and 2 |
| | N-terminus | |
| LMW-uPA-N 16 | 16 amino acid deletion from the | 9 and 2 |
| | N-terminus | |
| LMW-uPA-C 5 | 5 amino acid deletion from the | 10 and 1 |
| | C-terminus | |
| LMW-uPA-C 6 | 6 amino acid deletion from the | 11 and 1 |
| | C-terminus | |
| LMW-uPA-C 7 | 7 amino acid deletion from the | 12 and 1 |
| | C-terminus | |

All mutant constructs were expressed in E. coli as described above and the resulting synthesized polypeptides were shown to have similar activity to that of LMW-uPA in zymographic assays. The results of these experiments indicated that a functional modified uPA could be made consisting of amino acids 159-404 of human uPA with Cys²⁷⁹ replaced by Ala.

### EXAMPLE 2

### Cloning and Expression of micro-uPA[uPA(I159-K404)A279Q302] in Baculovirus

Micro-uPA (i.e. truncated uPA containing amino acids Ile¹⁵⁹-Lys⁴⁰⁴ and having substitutions of Ala for Cys²⁷⁹ and Gln for Asn³⁰² in SEQ ID NO: 1) was generated by PCR using the following oligonucleotide primers:

| | |
|---|---|
| SEQ ID NO: | SEQUENCE OF PCR PRIMER |

| | |
|---|---|
| 13 | 5′-ATTAATCAGCTGCTCCGGATAGAGATAGTCGGTAGACTGCTCTTTT-3′ |
| 14 | 5′-ATTAATCAGCTGAAAATGACTGTTGTGA-3′ |
| 15 | 5′-ATTAATGTCGACTAAGGAGGTGATCTAATGTTAAAATTTCAGTGTGGCCAA-3′ |
| 16 | 5′-ATTAATGCTAGCCTCGAGCCACCATGAGAGCCCTGCT-3′ |
| 17 | 5′-ATTAATGCTAGCCTCGAGTCACTTGTTGTGACTGCGGATCCA-3′ |
| 18 | 5′-GGTGGTGAATTCTCCCCCAATAATGCCTTTGGAGTCGCTCACGA-3′ |

To mutate the only glycosylation site (Asn³⁰²) in uPA, oligonucleotide primers SEQ ID NOs: 13 and 15, and SEQ ID NOs: 14 and 17 were used in two PCR reactions with pBC-LMW-uPA-Ala²⁷⁹ as the template. The two PCR products were cut with the restriction enzyme PvuII, ligated with T4 DNA ligase, and used as template to generate LMW-uPA-Ala ²⁷⁹-Gln³⁰². Native uPA leader sequence was fused directly to Ile¹⁵⁹ by PCR with SEQ ID NOs: 16 and 18 using native uPA cDNA as the template. This PCR product was used as a primer, together with SEQ ID NO: 17, in a new PCR reaction with LMW-uPA-Ala ²⁷⁹-Gln³⁰² DNA as template to generate micro-uPA cDNA. Micro-uPA was cut with Nhe I and ligated to a baculovirus transfer vector pJVP10z (Vialard et al., J. Virology, 64(1): 37-50 [1990]) cut with the same enzyme. The resulting construct, pJVP10z-micro-uPA, was confirmed by a standard DNA sequencing techniques.

Construct pJVP10z-micro-uPA was transfected into Sf9 cells by the calcium phosphate precipitation method using the BaculoGold kit from PharMingen (San Diego, Calif.). Active micro-uPA activity was detected in the culture medium. Single recombinant virus expressing micro-uPA was plaque purified by standard methods, and a large stock of the virus was made.

Large scale expression of micro-uPA was performed in another line of insect cells, High-Five cells (Invitrogen, Carlsbad, Calif.), in suspension, growing in Excel 405 serum free medium (JRH Biosciences, LeneXa, Kans.) in 2 liter flasks, with shaking at 80 rpm and at a temperature of 28° C. High-Five cells were grown to 2×10⁶ cells/mL, recombinant micro-uPA virus was added at 0.1 MOI (multiplicity of infection), and the culture was continued for 3 days. The culture supernatant was harvested as the starting material for purification (see Example 4 below). The activity of micro-uPA in the culture supernatant was measured by amidolysis of a chromogenic uPA substrate S2444 (Claeson et al., Haemostasis, 7: 76, 1978), which was at 6-10 mg/L.

### EXAMPLE 3

### Expression of micro-uPA in Pichia pastoris

To express micro-uPA in Pichia, an expression vector with a synthetic leader sequence (as described in U.S. Ser. No. 08/851,350, filed May 5, 1997 ) was used. The Pichia expression vector, pHil-D8, was constructed by modification of vector pHil-D2 (Invitrogen) to include a synthetic leader sequence for secretion of a recombinant protein. The leader sequence, SEQ ID NO: 19, (shown below) encodes a PHO1 secretion signal (single underline) operatively linked to a pro-peptide sequence (bold highlight) for KEX2 cleavage. To construct pHil-D8, PCR was performed using pHil-S1 (Invitrogen) as template since this vector contains the sequence encoding PHO1, a forward primer (SEQ ID NO: 20) corresponding to nucleotides 509-530 of pHil-S1 and a reverse primer (SEQ ID NO: 21) having a nucleotide sequence which encodes the latter portion of the PHO1 secretion signal (nucleotides 45-66 of SEQ ID NO: 19) and the pro-peptide sequence (nucleotides 67-108 of SEQ ID NO: 19). The primer sequences (obtained from Operon Technologies, Inc. Alameda, Calif.) were as follows:

| | |
|---|---|
| SEQ ID NO: | SEQUENCE OF PCR PRIMER |

| | |
|---|---|
| 19 | 5′-ATGTTCTCTCCAATTTTGTCCTTGGAAATTATTTTAGCTTTGGCTACTTTGCA |
| | ATCTGTCTTCGCTCAGCCAGTTATCTGCACTACCGTTGGTTCCGCTGCCG |
| | AGGGATCC-3′ |
| 20 | 5′-GAAACTTCCAAAAGTCGCCATA-3′ |
| 21 | 5′-ATTAATGATTCCTCGAGCGGTCCGGGATCCCTCGGCAGCGGAACCAACGGTA |
| | GTGCAGATAACTGGCTGAGCGAAGACAGATTGCAAAGTA-3′ |

Amplification was performed under standard PCR conditions. The PCR product (approximately 500 bp) was gel-purified, cut with BlpI and EcoRI and ligated to pHil-D2 cut with the same enzymes. The DNA was transformed into E. coli HB 101 cells and positive clones identified by restriction enzyme digestion and sequence analysis. One clone having the proper sequence was designated as pHil-D8.

The following two oligonucleotide primers then were used to amplify micro-uPA for cloning into pHil-D8.

| | |
|---|---|
| SEQ ID NO: | SEQUENCE OF PCR PRIMER |

| | |
|---|---|
| 22 | 5′-ATTAATGGATCCTTGGACAAGAGGATTATTGGGGGAGAATTCACCA-3′ |
| 23 | 5′-ATTAATCTCGAGCGGTCCGTCACTTGGTGTGACTGCGAATCCAGGGT-3′ |

The PCR product was obtained with SEQ ID NOs: 22 and 23 using pJVP10z-micro-uPA as the template. The amplified product was cut with BamHI and XhoI and ligated to pHil-D8 cut with the same two enzymes. The resulting plasmid, pHil-D8-micro-uPA, was confirmed by DNA sequencing, and used to transform a Pichia strain GS115 (Invitrogen) according to the supplier's instructions. Transformed Pichia colonies were screened for micro-uPA expression by growing in BMGY medium and expressing in BMMY medium as detailed by the supplier (Invitrogen). The micro-uPA activity was measured with chromogenic substrate S2444. The micro-uPA expression level in Pichia was higher than that seen in baculovirus-High Five cells, ranging from 30-60 mg/L.

### EXAMPLE 4

### Purification of micro-uPA

There are two suitable methods capable of purifying u-PA within the scope of the invention, described below as 4a. and 4b.

4a. The culture supernant of either High Five cells or Pichia were pooled into a 20 liter container. Protease inhibitors iodoacetamide, benzamidine and EDTA were added to final concentrations of 10 mM, 5 mM and 1 mM, respectively. The supernatant was then diluted 5-fold by adding 5 mM Hepes buffer pH7.5 and passed through 1.2μ and 0.2μ filter membranes. The micro-uPA was captured onto Sartorius membrane adsorber S100 (Sartorius, Edgewood, N.Y.) by passing through the membrane at a flow rate of 50-100 mL/min. After extensive washing with 10 mM Hepes buffer, pH7.5, containing 10 mM iodoacetamide, 5 mM benzamidine, 1 mM EDTA, micro-uPA was eluted from S100 membrane with a NaCl gradient (20 mM to 500 mM, 200 mL) in 10 mM Hepes buffer, pH7.5, 10 mM iodoacetamide, 5 mM benzamidine, 1 mM EDTA. The eluate (˜100ml) was diluted 10 times in 10 mM Hepes buffer containing inhibitors, and loaded onto a S20 column (BioRad, Hercules, Calif.). Micro-uPA was eluted with a 20× column volume NaCl gradient (20 mM to 500 mM). No inhibitors were used in the elution buffers. The eluate was then diluted 5-fold with 10 mM Hepes buffer, pH7.5, and loaded to a heparin-agarose (SIGMA, St. Louis, Mo.) column. Micro-uPA was eluted with a NaCl gradient from 10 mM to 250 mM. The heparin column eluate of micro-uPA (˜50 mL) was applied to a Benzamidine-agarose (SIGMA) column (40 mL) equilkibrated with 10 mM Hepes buffer, pH7.5, 200 mM NaCl. The column was then washed the equilibration buffer and eluted with 50 mM NaOAc, pH 4.5, 500 mM NaCl. The micro-uPA eluate (˜30 mL) was concentrated to 4 mL by ultrafiltration and applied to a Sephadex® G-75 column (2.5×48 cm, Pharmacia® Biotech, Uppsala, Sweden) equilibrated with 20 mM NaOAc, pH4.5, 100 mM NaCl. The single major peak containing micro-uPA was collected and lyophilized as the final product. The purified material appeared on SDS-PAGE as a single major band.

4b. Step 1. Capture of mUK from the conditioned medium.

Either of two alternative steps may be used for the initial capture. The choice is a matter of scale. For small scale purifications the mUK may be captured using hydrophobic interaction chromatography such as HiPropyl (J. T. Baker) or equivalent, and for larger scale purifications it may be captured by cation exchange chromatography using an S-Sepharose Fast Flo resin(Pharmacia Biotech) or equivalent.

For the small scale process, the ionic strength of the medium is increased by the addition of a particular volume of 4.5M sodium acetate pH7.0 to give a final solution of 1.1M sodium acetate in the final volume. This sample is applied to a HiPropyl column previously equilibrated in 1.1M sodium acetate pH7.0. In this manner, the desired mUK is bound to the column and other proteins are not bound. The non-bound proteins are washed out of the column by rinsing with at least 5 column volumes of 1.1M sodium acetate pH7.0 containing 1 mM p-aminobenzamidine (pABA). The mUK is released from the column by developing a gradient in 10 column volumes to buffer B which is 50 mM Tris, 0.2M NaCl, 1 mM pABA. The location of the mUK in the gradient is found by enzymatic assay of the collected fractions and is confirmed by SDS-PAGE. From this a pool of fractions is made which is dialyzed against 10 volumes of buffer C (50 mM Tris, 0.5M NaCl, 1 mM pABA, pH7.5) in preparation for Step 2.

For the large scale process, the ionic strength of the medium is decreased by dilution into water, and the pH is adjusted to the range pH5.0 to pH5.5 by the addition of 10 mM MES pH5.0 (buffer D), if necessary. This diluted sample is applied to an S-Sepharose FastFlo column previously equilibrated in buffer D. In this manner, the desired mUK is bound to the column and other proteins are not bound. The mUK is eluted from the column by developing a 10 column volume gradient with 1M NaCl in buffer D. The location of the mUK in the gradient is found by enzymatic assay of the collected fractions and is confirmed by SDS-PAGE. From this a pool of fractions is made which is dialyzed against 10 volumes of buffer C (50 mM Tris, 0.5M NaCl, 1 mM pABA, pH7.5) in preparation for Step 2.

Step 2. Removal of carbohydrate modified forms of mUK.

The dialyzed material from Step 1 is applied to a ConcanavalinA-Sepharose (Pharmacia Biotech) column previously equilibrated in buffer C. The column flow is slow to allow sufficient time and the column volume is large to provide sufficient capacity to bind the glycosylated forms of mUK to the resin and allow the desired non-glycosylated form of mUK to flow through the column. The location of this desired mUK that is not bound to the column is found by enzymatic assay of the collected fractions and is confirmed by SDS-PAGE.

Step 3. Dialysis to remove pABA.

The pool of fractions from Step 2 is adjusted to pH5.0 by addition of 2M sodium acetate pH4.5. This pool is twice dialyzed at 4C against 100 volumes of 10 mM MES, 0.5M NaCl pH5.0 with one change of the dialysate after several hours such that the concentration of pABA is greatly decreased overnight. After the dialysis is ended and immediately before step 4, the pH of the dialysate is raised to pH 7.5 by the addition of 1M Tris base pH8.0.

Step 4. Affinity selection of intact, active mUK on Benzamidine-Sepharose.

The mUK in the pH adjusted dialysate contains intact, active molecules of mUK as well as less active, partially damaged forms of UK that have lower affinity for the Benzamidine-Sepharose (Pharmacia Biotech). The pH7.5 dialysate from Step 5 is applied to an Benzamidine-Sepharose affinity column so that the active mUK will bind to the column previously equilibrated in 50 mM Tris, 0.5M NaCl pH7.5 (buffer E). Non-bound proteins are washed out of the column with 1.5 column volumes of buffer E, after which the intact, active UK is eluted with a 10 column volume gradient of 1M arginine in buffer E, re-adjusted to pH7.5. During the development of the gradient, damaged molecules of mUK elute earlier in the gradient than intact, active molecules. The location of the intact, active mUK that is found by enzymatic assay of the collected fractions and is confirmed by SDS-PAGE.

Step 5. Removal of arginine by dialysis.

The pool of intact, active UK is twice dialyzed at 4C against 100 volumes of 50 mm sodium acetate pH4.5 with one change after several hours such that the concentration of arginine is greatly decreased overnight.

### EXAMPLE 5

### Co-crystallization of Micro-uPA

a. Methods: Micro-uPA was crystallized by the hanging drop vapor diffusion method, (essentially as described in U.S. Pat. No. 4,886,646, issued Dec. 12, 1989) in the presence of an inhibitor, ε-amino caproic acid p-carbethoxyphenyl ester chloride described by Menigath et al. (J. Enzyme Inhibition, 2: 249-259 [1989]). The protein solution consisted of 6 mg/mL (0.214 mM) micro-uPA in 10 mM citrate pH 4.0 and 3 mM ε-amino caproic acid p-carbethoxyphenyl ester chloride in 1% DMSO co-solvent. In making the protein solution, the inhibitor (300-400 mM DMSO stock solution) was added to the micro-uPA to a final inhibitor concentration of approximately 3 mM (1% DMSO). Typical well solutions consisted of 0.15M Li₂SO₄, 20% polyethylene glycol (MW 4000) and succinate buffer (pH 4.8-6.0). On the cover slip, well solution (2 μL) was mixed with protein solution (2 μL) and the slip sealed over the well. Crystals were grown in Linbro trays (Hampton Research, San Franscisco, Calif.) at 18-24 ° C. Under these conditions, crystallization occurred within 24 hours.

Because micro-uPA will not crystallize in absence of an inhibitor, the co-crystallizing entity is believed to be the inhibitor:uPA complex. As a theory, it is believed that the inhibitor used in the co-crystallizing procedure is meta-stable, i.e. that it acylates the active site serine (amino acid residue 356 of SEQ ID NO: 1) and is subsequently deacylated enzymatically, because, the 3-D X-ray structure of crystals grown in the presence of this compound shows no inhibitor remaining in the enzyme active site. Although the actual mechanism by which the inhibitor dissociates from the crystal is unknown, the resultant micro-uPA crystals are composed of enzyme with an empty active site.

b. Results: Crystals obtained under the conditions described above belong to the space group P2₁2₁2₁ with unit cell dimensions of a=55.16Å, b-53.00Å, c=82.30Å, and α=β=γ=90. They diffract to beyond 1.5Å in house and a 1.03Å resolution native data set was collected on a CCD detector at the Cornell High Energy Synchrotron Source in Ithaca, N.Y. Data were processed by the program package DENZO (Otwinowski and Mino, Methods in Enzymology 276, 1996). Parameters summarizing data quality for the 1.03Å data set are summarized in Table 1 below. Table 1 shows that data were 85.9% complete in the data shell from 1.04-1.0Å resolution with an I/σ of 1.78 although the merging Rsym was high at 0.631. Hence the data incorporated into the refinement cycles were cut at 1.04Å because in the 1.08-1.04Å data shell the Rsym was 0.463 with an I/σ of 2.67.

TABLE 1
Diffraction Data Quality Statistics

| | No. Unique | | | |
|---|---|---|---|---|
| | Reflections | % Complete | I/σ | Rsym (square) |
| overall | 108878 | 91.3 | 16.5 | 0.089 |
| 1.08-1.04Å | 10347 | 87.9 | 2.67 | 0.463 |
| 1.04-1.00Å | 10157 | 85.9 | 1.78 | 0.631 |

Phases were determined by the molecular replacement method using the program AMORE (Navaza, J. Acta Cryst., A50: 157-163 [1994]) with the urokinase structure of Spraggon et al. (Structure 3: 681-691 (1995), PDB entry 1 LMW) being used as the search probe. The rotation and translation functions were performed using data between 5 and 30Å resolution with the correct solution being among the top peaks. The structure was refined using the program package XPLOR by a combination of rigid body, simulated annealing maximum likelihood refinement, and maximum likelihood positional refinement (Brunger, A. X-PLOR (version 2.1) Manual, Yale University, New Haven, Conn., 1990). Electron density maps were inspected on a Silicon Graphics INDIGO2 workstation using the program package QUANTA 97 (Molecular Simulations Inc., Quanta Generating and Displaying Molecules, San Diego: Molecular Simulations Inc., 1997). Cycles of model building of the protein structure occurred at 2.0Å resolution, 1.5Å resolution and 1.03Å resolution. At 1.03Å resolution constrained individual temperature factor refinement was also included in the refinement cycle. Following model building and the addition of alternate side chain conformations, cycles of water molecule and bound ion addition also occurred through the identification of positive peaks in the Fo-Fc map at least 4σ above noise. The R-factor of the current model is 0.233 and the R-free is 0.287.

We claim:

1. A polynucleotide which encodes a biologically active modified urinary-type plasminogen activator (mod-uPA) having at least 70% identity to an amino acid sequence selected from the group consisting of
(a) amino acid position 159 to amino acid position 404 of SEQ ID NO: 1;
(b) amino acid position 159 to amino acid position 405 of SEQ ID NO: 1;
(c) amino acid position 159 to amino acid position 406 of SEQ ID NO: 1;
(d) amino acid position 159 to amino acid position 407 of SEQ ID NO: 1;
(e) amino acid position 159 to amino acid position 408 of SEQ ID NO: 1;
(f) amino acid position 159 to amino acid position 409 of SEQ ID NO: 1;
(g) amino acid position 159 to amino acid position 410 of SEQ ID NO: 1; and
(h) from amino acid position 159 to amino acid position 411 of SEQ ID NO: 1;
wherein amino acid residues at positions 279 and 302 (Xaa²⁷⁹ and Xaa³⁰²) are any amino acids.

2. The polynucleotide of claim 1 wherein said Xaa²⁷⁹ residue is Ala.

3. The polynucleotide of claim 2 wherein said Xaa³⁰² residue is Gln.

4. A recombinant vector comprising the polynucleotide of claim 1.

5. A recombinant vector comprising the polynucleotide of claim 2.

6. A recombinant vector comprising the polynucleotide of claim 3.

7. A recombinant vector of claim 5 which is a baculovirus vector.

8. The recombinant vector of claim 3 which is a baculovirus vector.

9. A host cell comprising the vector of claim 4.

10. A host cell comprising the vector of claim 5.

11. A host cell comprising the vector of claim 6.

12. A biologically active modified urinary-type plasminogen activator (mod-uPA) having at least 70% identity to an amino acid sequence selected from the group consisting of
(a) amino acid position 159 to about amino acid position 404 of SEQ ID NO: 1;
(b) amino acid position 159 to amino acid position 405 of SEQ ID NO: 1;
(c) amino acid position 159 to amino acid position 406 of SEQ ID NO: 1;
(d) amino acid position 159 to amino acid position 407 of SEQ ID NO: 1;
(e) amino acid position 159 to amino acid position 408 of SEQ ID NO: 1;
(f) amino acid position 159 to amino acid position 409 of SEQ ID NO: 1;
(g) amino acid position 159 to amino acid position 410 of SEQ ID NO: 1;
and
(h) from amino acid position 159 to amino acid position 411 of SEQ ID NO: 1;
with the proviso that when said mod-uPA is glycosylated, residue 279 is any amino acid residue other than Cys and when said mod-uPA is non-glycosylated, residue 279 is any amino acid.

13. The mod-uPA of claim 12 wherein said Xaa residue at position 279 is Ala.

14. The mod-uPA of claim 13 wherein said Xaa residue at position 302 is Gln.

15. A crystalline form of mod-uPA wherein the primary structure of said mod-uPA has at least 70% identity to an amino acid sequence selected from the group consisting of
(a) amino acid position 159 to about amino acid position 404 of SEQ ID NO: 1;
(b) amino acid position 159 to amino acid position 405 of SEQ ID NO: 1;
(c) amino acid position 159 to amino acid position 406 of SEQ ID NO: 1;
(d) amino acid position 159 to amino acid position 407 of SEQ ID NO: 1;
(e) amino acid position 159 to amino acid position 408 of SEQ ID NO: 1;
(f) amino acid position 159 to amino acid position 409 of SEQ ID NO: 1;
(g) amino acid position 159 to amino acid position 410 of SEQ ID NO: 1; and
(h) from amino acid position 159 to amino acid position 411 of SEQ ID NO: 1;
with the proviso that when said mod-uPA is glycosylated, residue 279 is any amino acid residue other than Cys and when said mod-uPA is non-glycosylated, residue 279 is any amino acid.

16. The crystalline mod-uPA of claim 15 wherein Xaa residue at position 279 is Ala.

17. The crystalline mod-uPA of claim 16 wherein said Xaa residue at position 302 is Gln.

18. A method for making mod-uPA comprising the steps of:
(a) culturing the host cell of claim 4 under conditions that allow the production of the mod-uPA polypeptide; and
(b) recovering the mod-uPA polypeptide.

19. A method for making mod-uPA comprising the steps of:
(a) culturing the host cell of claim 5 under conditions that allow the production of the mod-uPA polypeptide; and
(b) recovering the mod-uPA polypeptide.

20. A method for making mod-uPA comprising the steps of:
(a) culturing the host cell of claim 6 under conditions that allow the production of the mod-uPA polypeptide; and
(b) recovering the mod-uPA polypeptide.
